(19) United States
(12) Patent Application Publication
(10) Pub. No.: US 2026/0144900 A1
(43) Pub. Date: May 28, 2026

(54) IN VIVO CROSSLINKABLE HYDROGELS WITH GOLD NANOPARTICLES FOR MEDICAL APPLICATIONS

(71) Applicant: Boston Scientific Scimed, Inc., Maple Grove, MN (US)

(72) Inventors: Rick William Dorn, Plymouth, MN (US); Joseph Thomas Delaney, JR., Minneapolis, MN (US)

(73) Assignee: Boston Scientific Scimed, Inc., Maple Grove, MN (US)

(21) Appl. No.: 19/177,857

(22) Filed: Apr. 14, 2025

Related U.S. Application Data
(60) Provisional application No. 63/633,927, filed on Apr. 15, 2024.

(51) Int. Cl. A61K 49/04

(52) U.S. Cl. CPC A61K 49/049; A61K 49/0457

(57) ABSTRACT

In some aspects, the present disclosure pertains to systems that comprise (a) a reactive polymer comprising a plurality of first hydrophilic polymer segments and a plurality of first reactive moieties, (b) a reactive multifunctional compound comprising a plurality of second reactive moieties, and (c) gold nanoparticles that are functionalized with a plurality of second hydrophilic polymer segments. In other aspects, the present disclosure pertains to crosslinked reaction products formed from such systems. In further aspects, the present disclosure pertains to methods of treatment using systems and such crosslinked reaction products.

### CROSS-REFERENCE TO RELATED APPLICATIONS

This application claims the benefit of U.S. Provisional Patent Application Ser. No. 63/633,927 filed on Apr. 15, 2024, the disclosure of which is incorporated herein by reference.

### FIELD

The present disclosure relates to radiopaque hydrogels and to crosslinkable systems for forming radiopaque hydrogels, among other aspects. The radiopaque hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups rapidly react with the trilysine crosslinker in vivo to form a hydrogel. The hydrogels breakdown in-vivo over the course of circa 6-9 months. The breakdown occurs primarily through the hydrolysis of the ester linkages on the glutarate groups.

### SUMMARY

The present disclosure provides alternative systems for use in forming hydrogels, including iodine-free hydrogels, while maintaining or improving crosslink density and/or radiopacity. As used herein, a “hydrogel,” also referred to herein as a “crosslinked hydrogel,” is a crosslinked polymer that contains water or can absorb water but does not dissolve when placed in water.

In some aspects, the present disclosure pertains to systems that comprise (a) a reactive polymer comprising a plurality of first hydrophilic polymer segments and a plurality of first reactive moieties, (b) a reactive multifunctional compound comprising a plurality of second reactive moieties, and (c) gold nanoparticles that are functionalized with a plurality of second hydrophilic polymer segments.

In some embodiments, the reactive polymer is a multi-arm polymer that comprises three or more polymer arms linked to a core region, each arm comprising one of the first hydrophilic polymer segments and one of the first reactive moieties.

In some embodiments, which can be used in conjunction with the above aspects and embodiments, the reactive polymer is a multi-arm polymer that comprises three or more polymer arms linked to a core region, each arm comprising a cyclic anhydride residue disposed between a first hydrophilic polymer segment and a first reactive moiety.

In some embodiments, which can be used in conjunction with the above aspects and embodiments, the first hydrophilic polymer segments are selected from poly(alkylene oxide) segments, polysaccharide segments, polyoxazoline segments, polydioxanone segments, polypeptide segments, and polyvinyl alcohol segments.

In some embodiments, which can be used in conjunction with the above aspects and embodiments, each of the first hydrophilic polymer segments contains between 10 and 1000 monomer residues.

In some embodiments, which can be used in conjunction with the above aspects and embodiments, the gold nanoparticles range from 1 nm to 2 micrometers in longest dimension.

In some embodiments, which can be used in conjunction with the above aspects and embodiments, the second hydrophilic polymer segments are selected from poly(alkylene oxide) segments, polysaccharide segments, polyoxazoline segments, polydioxanone segments, polypeptide segments, and polyvinyl alcohol segments.

In some embodiments, which can be used in conjunction with the above aspects and embodiments, the second reactive moieties are attached to a polyol residue or a polycarboxylic acid residue.

In some embodiments, which can be used in conjunction with the above aspects and embodiments, the second reactive moieties are attached to the multifunctional compound through a hydrolysable ester group.

In some embodiments, which can be used in conjunction with the above aspects and embodiments, the first reactive moieties comprise a cyclic imide ester group and the second reactive moieties comprise a primary amine, thiol or hydroxyl group.

In some embodiments, which can be used in conjunction with the above aspects and embodiments, the first reactive moieties comprise a primary amine, thiol or hydroxyl group and the second reactive moieties comprise a cyclic imide ester group.

In some embodiments, which can be used in conjunction with the above aspects and embodiments, the first reactive moieties comprise a strained alkyne group and the second reactive moieties comprise an azide group.

In some embodiments, which can be used in conjunction with the above aspects and embodiments, the first reactive moieties comprise an azide group and the second reactive moieties comprise a strained alkyne group.

In some embodiments, which can be used in conjunction with the above aspects and embodiments, the first reactive moieties comprise a strained alkene group and the second reactive moieties comprise tetrazine a group.

In some embodiments, which can be used in conjunction with the above aspects and embodiments, the first reactive moieties comprise a tetrazine group and the second reactive moieties comprise a strained alkene group.

In some embodiments, which can be used in conjunction with the above aspects and embodiments, the system further comprises a delivery device.

In other aspects, the present disclosure provides method of treatment comprising administering to a subject a mixture that comprises a reactive polymer, a multifunctional compound, and gold nanoparticles in accordance with any of the above aspects and embodiments, under conditions such that the reactive polymer and the multifunctional compound crosslink after administration to form a hydrogel in which the gold nanoparticles are retained in the hydrogel via non-covalent interactions.

In some embodiments, the methods comprise administering to the subject a first fluid composition that comprises the reactive polymer and a second fluid composition that comprises the multifunctional compound, wherein the gold nanoparticles are provided in the first fluid composition, the second composition, or both.

In some embodiments, the methods comprise administering to the subject a first fluid composition that comprises the reactive polymer and the multifunctional compound and a second fluid composition that comprises an accelerant that accelerates formation of the covalent crosslinks, wherein the gold nanoparticles are provided in the first fluid composition, the second composition, or both.

In some of these embodiments, the first fluid composition and the second fluid composition are delivered using a double barrel syringe.

In other aspects, the present disclosure pertains to crosslinked hydrogels in which gold nanoparticles in accordance with any of any of the above aspects and embodiments are retained in a crosslinked reaction product of a reactive polymer and a multifunctional compound in accordance with any of any of the above aspects and embodiments.

In some embodiments, the crosslinked hydrogels are in the form of injectable particles.

In some embodiments, which can be used in conjunction with the above aspects and embodiments, the crosslinked hydrogels comprise hydrolysable linkages, and the crosslinked hydrogels bioerode in vivo over a period ranging from 1 day or less to 5 years or longer, for example, ranging anywhere from 1 day to 3 days to 1 week to 2 weeks to 1 month to 6 months to 1 year to 2 years to 5 years of longer (in other words, over a period ranging between any two of the preceding values).

In other aspects, the present disclosure pertains to methods of treatment comprising administering to a subject the radiopaque crosslinked hydrogel compositions of any of the above aspects and embodiments.

The above and other aspects, embodiments, features and benefits of the present disclosure will be readily apparent from the following detailed description.

### BRIEF DESCRIPTION OF THE DRAWINGS

FIG. 1 schematically illustrates a reactive multi-arm polymer, in accordance with an embodiment of the present disclosure.

FIG. 2 schematically illustrates a method of forming a functionalized gold nanoparticle, in accordance with an embodiment of the present disclosure.

FIGS. 3A-3D schematically illustrate methods of forming reactive polymers, in accordance with four embodiments of the present disclosure.

FIGS. 4A-4C schematically illustrate methods of forming covalent linkages, in accordance with three embodiments of the present disclosure.

FIG. 5 schematically illustrates a method of forming a crosslinked product by covalently linking a reactive multi-arm polymer and a reactive multifunctional compound in the presence of functionalized gold nanoparticles, in accordance with an embodiment of the present disclosure.

FIG. 6 illustrates a delivery device, in accordance with an embodiment of the present disclosure.

FIG. 7 illustrates a delivery device, in accordance with another embodiment of the present disclosure.

### DETAILED DESCRIPTION

In some embodiments, the system comprises (a) a reactive polymer comprising a plurality of first hydrophilic polymer segments and a plurality of first reactive moieties, (b) a reactive multifunctional compound comprising a plurality of second reactive moieties, and (c) gold nanoparticles that are functionalized with polymers having second hydrophilic polymer segments.

In various embodiments, the system is configured to deliver the reactive polymer, the reactive multifunctional compound, and the gold nanoparticles under conditions such that covalent crosslinks are formed between the first reactive moieties of the reactive polymer and the second reactive moieties of the reactive multifunctional compound, and a hydrogel is formed in which the gold nanoparticles are entrapped.

Particular examples of first and second reactive moieties include the following among others (a) first reactive moieties that comprise electrophilic groups and second reactive moieties that comprise nucleophilic groups, or vice versa, (b) first reactive moieties that comprise strained alkyne groups and second reactive moieties that comprise azide groups, or vice versa, and (c) first reactive moieties that comprise strained alkene groups and second reactive moieties that comprise tetrazine groups, or vice versa.

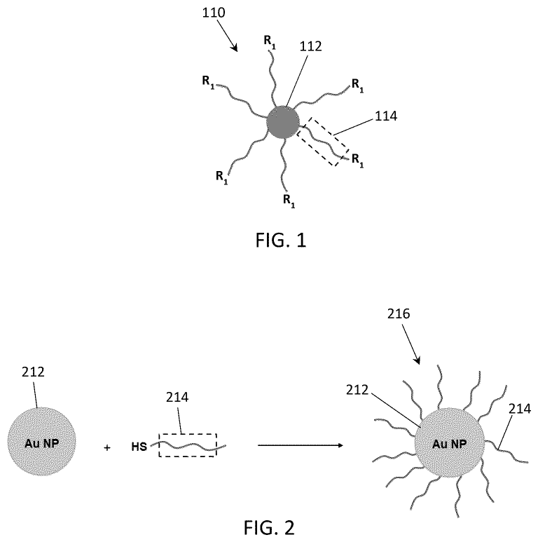

Referring now to FIG. 1, reactive polymers in accordance with the present disclosure include reactive multi-arm polymers 110 that comprise a plurality of polymer arms linked to a core region 112, at least a portion of the polymer arms comprising a hydrophilic polymer segment 114. One end of the hydrophilic polymer segment 114 is covalently linked to the core region 112 through a suitable linkage and an opposite end of the hydrophilic polymer segment 114 is covalently linked to a first reactive moiety R₁ through a suitable linkage.

In some embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a first reactive moiety that is covalently linked to the second end of the cyclic anhydride residue.

Reactive polymers in accordance with the present disclosure include polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

First reactive moieties R₁ include moieties that comprise electrophilic groups, moieties that comprise nucleophilic groups, moieties that comprise strained alkyne groups, moieties that comprise strained alkene groups, moieties that comprise azide groups,

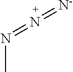

and moieties that comprise tetrazine groups

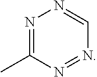

Electrophilic groups may be selected, for example, from cyclic imide ester groups, such as succinimide ester groups,

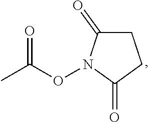

maleimide ester groups, glutarimide ester groups, diglycolimide ester groups, phthalimide ester groups, and bicyclo[2.2.1]hept-5-ene-2,3-dicarboxylic acid imide ester groups,

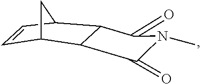

imidazole ester groups, imidazole carboxylate groups and benzotriazole ester groups, among other possibilities. Nucleophilic groups may be selected, for example, from amine groups, thiol groups, and hydroxyl groups, among other possibilities. Strained alkyne groups may be selected, for example, from (1R,8S,9s)-bicyclo[6.1.0]non-4-yn-9-yl groups,

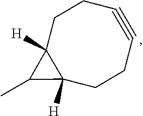

and dibenzocyclooctyne groups, among other possibilities. Strained alkene groups may be selected, for example, from cyclooct-4-en-1-yl groups,

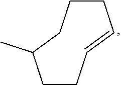

cyclooct-4-enyl 2,5-dioxopyrrolidin-1-yl carbonate groups, carbamic acid, N-(3-aminopropyl)-, 4-cycloocten-1-yl ester groups, or carbamic acid, N-[2-[2-(2-aminoethoxy) ethoxy]ethyl]-, 4-cycloocten-1-yl ester groups, among other possibilities.

The electrophilic groups, nucleophilic groups, strained alkyne groups, strained alkene groups, azide groups, or tetrazine groups may be linked to the hydrophilic polymer segment 114 through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, or a linking moiety that comprises a combination of two or more of the foregoing groups, among others. In various embodiments, the linking moiety comprises a hydrolysable ester group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging from 5 to 10 to 20 to 50 to 100 to 200 to 500 to 1000 to 2000 monomer units or more.

In certain embodiments, the core region comprises a residue of a polyhydroxy compound comprising three or more hydroxyl groups, also referred to herein as a “polyol”, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RsiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The Ts cage-like silicon-oxygen cores are widely studied and have the formula [RsiO_{3/2}]₈,

or equivalently R₈Si₈O₁₂. Such a structure is shown here:

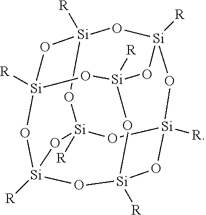

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some of these embodiments, the hydroxy-terminated precursor multi-arm hydrophilic polymer may be reacted with a cyclic anhydride to form an acid-end-capped precursor polymer in which carboxylic acid end groups are linked to hydrophilic polymer segments through hydrolysable ester groups. For example, terminal hydroxyl groups of the hydrophilic polymer segments may be reacted with a cyclic anhydride (e.g., glutaric anhydride, succinic anhydride, malonic anhydride, adipic anhydride, diglycolic anhydride, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group.

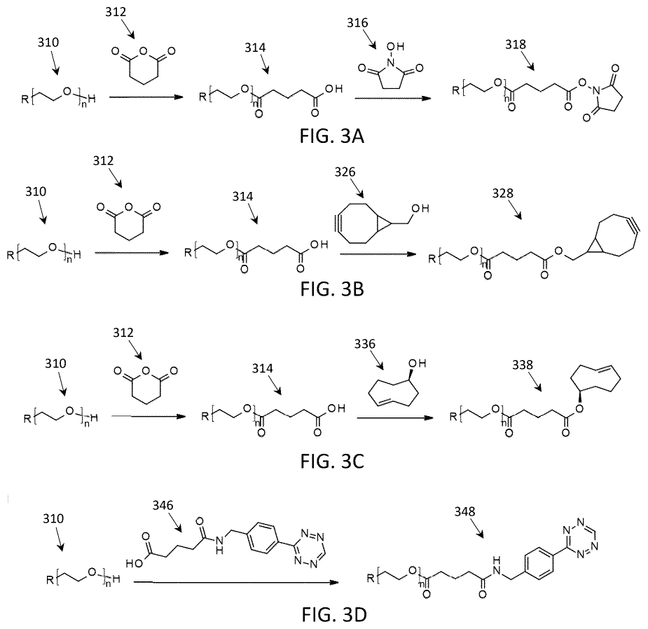

With reference now to FIGS. 3A-3C, a cyclic anhydride, specifically glutaric anhydride 312, is reacted with a hydroxy-terminated precursor multi-arm hydrophilic polymer, specifically a hydroxy-terminated precursor multi-arm polyethylene oxide (PEO) 310, where R corresponds to a core, to form an acid end-capped multi-arm polymer, specifically glutaric-acid-end-capped multi-arm polyethylene oxide (PEO) 314. (It is noted that although only one arm of the multi-arm polyethylene oxide 310 is shown attached to the core R in FIGS. 3A-3C, as well as in FIG. 3D described below, it is to be understood that additional polymer arms are present.) In FIGS. 3A-3D, n is an integer and may have a value ranging from 5 to 1000 or more.

A reactive moiety may then be linked to the carboxylic-acid-terminated precursor polymer. In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include malcimidyl malonate groups, malcimidyl glutarate groups, malcimidyl succinate groups, malcimidyl adipate groups, and malcimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In a particular embodiment shown in FIG. 3A, a glutaric acid end-capped multi-arm PEO 314 is reacted with N-hydroxysuccinimide 316 in the presence of a coupling agent to form a succinimidyl-glutarate-end-capped multi-arm PEO 318.

In some embodiments, primary amine groups may be linked to the carboxylic-acid-terminated precursor polymer. For example, a diamine compound such as 1,2-diaminocthane, 1,3-diaminoproane, 1,4-diaminobutane, etc. in which one of the amino groups is protected with a suitable protective group (e.g., a tert-butyloxycarbonyl (tBoc) or a fluorenylmethyloxycarbonyl (Fmoc) or a benzyloxycarbonyl (Cbz) protective group) can be reacted in an amide coupling reaction with carboxylic acid groups of a carboxylic-acid-terminated precursor polymer such as those described above (e.g., a glutaric acid end-capped multi-arm PEO 314 Illustrated in FIG. 3A) in the presence of a suitable coupling agent such as a carbodiimide coupling agent to form an amino-terminated polymer, for example, an amino-C₁-C₄-alkyl-terminated polymer in which the amino-C₁-C₄-alkyl groups are linked to the hydrophilic polymer segments through an amide group and a hydrolysable ester group.

In some embodiments, strained alkyne groups may be linked to the carboxylic-acid-terminated precursor polymer. In a particular embodiment shown in FIG. 3B, glutaric acid end-capped multi-arm PEO 314 is reacted with a hydroxyl-substituted strained alkyne such as (1R,8S,9s)-bicyclo[6.1.0]non-4-yn-9-ylmethanol 326 in an ester coupling reaction in the presence of a suitable coupling agent to produce (1R,8S,9s)-bicyclo[6.1.0]non-4-yn-9-yl-glutarate-end-capped multi-arm PEO 328, in which (1R,8S,9s)-bicyclo[6.1.0]non-4-yn-9-yl groups are coupled to the polymer arms through two hydrolysable ester groups. In an alternative embodiment (not shown), acid end-capped multi-arm PEO is reacted with an amine-substituted strained alkyne such as (1R,8S,9s)-bicyclo[6.1.0]non-4-yn-9-ylmethamine in an amide coupling reaction in the presence of a suitable coupling agent to produce a multi-arm PEO in which (1R,8S,9s)-bicyclo[6.1.0]non-4-yn-9-yl groups are coupled to the polymer arms through an amide group and a hydrolysable ester group.

In some embodiments, strained alkene groups may be linked to the carboxylic-acid-terminated precursor polymer. In a particular embodiment shown in FIG. 3C, glutaric acid end-capped multi-arm PEO 314 is reacted with a hydroxyl-substituted strained alkene such as cyclooct-4-en-1-ol 336 in an ester coupling reaction in the presence of a suitable coupling agent to produce cyclooct-4-en-1-yl-glutarate-end-capped multi-arm PEO 338, in which cyclooct-4-en-1-yl groups are coupled to the polymer arms through two hydrolysable ester groups. In an alternative embodiment (not shown), acid end-capped multi-arm PEO is reacted with an amine-substituted strained alkene such as cyclooct-4-en-1-amine in an amide coupling reaction in the presence of a suitable coupling agent to produce a multi-arm PEO in which cyclooct-4-en-1-yl groups are coupled to the polymer arms through an amide group and a hydrolysable ester group.

In some embodiments, tetrazine groups may be linked to a hydroxyl-terminated precursor polymer. Exemplary hydroxyl-terminated precursor polymers are described above and include the hydroxy-terminated precursor multi-arm polyethylene oxide (PEO) 310 of FIG. 3A, among many others. For example, a tetrazine based acid may be coupled to a hydroxyl-terminated precursor polymer in an ester coupling reaction in the presence of a suitable coupling agent. In a particular embodiment shown in FIG. 3D, a tetrazine-terminated polymer is prepared by coupling a hydroxy-terminated precursor multi-arm polyethylene oxide (PEO) 310 with 5-[4-(1,2,4,5-tetrazin-3-yl)benzylamino]-5-oxopentanoic acid in the presence of a carbodiimide coupling agent to produce 5-[4-(1,2,4,5-tetrazin-3-yl)benzylamino]-5-oxopentanoate-end-capped multi-arm PEO 338. It is noted that the reactive tetrazine groups are linked to the multi-arm polymer through a hydrolysable ester group.

In some embodiments, an azide-terminated polymer may be formed from a hydroxyl-terminated precursor polymer. For example, an azide-terminated polymer may be synthesized by reacting a hydroxy-terminated precursor multi-arm polymer with methanesulfonyl chloride to obtain a methanesulfonyl-terminated multi-arm polymer. Then, sodium azide (NaN₃) is reacted with the methanesulfonyl-terminated multi-arm polymer to replace the methanesulfonate groups with azide groups, forming an azide-terminated multi-arm polymer.

Exemplary hydroxyl-terminated precursor polymers are described above and include the hydroxy-terminated precursor multi-arm polyethylene oxide (PEO) 310 of FIG. 3A, among many others.

In embodiments where a hydrolysable ester linkage is desired as well as a hydroxyl end group, hydroxyl groups of a hydroxyl-terminated precursor polymer, such as the hydroxy-terminated precursor multi-arm polyethylene oxide (PEO) 310 of FIG. 3A, among many others, may be reacted in a ring-opening reaction with a lactone (e.g., α-acetolactone, β-propiolactone, γ-butyrolactone, δ-valerolactone, ε-caprolactone, etc.) to form a hydroxyl-terminated precursor polymer that comprises a hydroxyl end group that is linked to a residue of the hydroxyl-terminated precursor polymer through an alkyl group (e.g., C₁-C₁₀ alkyl group) and a hydrolysable ester group at the site of each of the hydroxyl groups of the hydroxyl-terminated precursor polymer.

The above strategies are widely applicable to hydroxy-terminated polymers having hydrophilic polymer segments other PEO segments, such as the segments disclosed above.

As previously indicated, in addition to a reactive polymer comprising a plurality of first hydrophilic polymer segments and a plurality of first reactive moieties as described above, the systems described herein further comprise a reactive multifunctional compound comprising a plurality of second reactive moieties, wherein the first reactive moieties and the second reactive moieties are selected to covalently crosslink with one another to form a crosslinked reaction product.

In some embodiments, the multifunctional compound is a compound having at two or more nucleophilic groups, for example, having 2, 3, 4, 5, 6, 7, 8, 9, 10, 11, 12, 13, 14, 15, 16, 17, 18, 19, 20 or more nucleophilic groups. Specific examples of such multifunctional compounds include, for example, polyamines that contain at two or more amino (—NH₂) groups (e.g., 2, 3, 4, 5, 6, 7, 8, 9, 10 or more amino groups in some embodiments), also referred to herein as polyamino compounds. Polyamino compounds suitable for use in the present disclosure include polyamino compounds that comprise two or more primary amine groups, for example, —(CH₂)ₓ—NH₂ groups where x is 1, 2, 3, 4, 5 or 6. Polyamino compounds suitable for use in the present disclosure include polyamino compounds that comprise two or more basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further examples of polyamino compounds which may be used as the multifunctional compound include ethylenediamine, ethylenetriamine, diethylene triamine, hexamethylenetriiamine, di(heptamethylene)triamine, di(trimethylene) triamine, bis(hexamethylene)triamine, tris(2-aminoethyl)amine, tris(3-aminopropyl)amine, 1,3,5-tris-(2-aminoethyl)-[1,3,5]triazinane-2,4,6-trione, N,N,N′-tris(2-aminoctbyl)ethylenediamine, triethylene tetramine, tripropylene tetramine, N,N′,N′-tetrakis(2-aminoethyl)-1,2-ethanediamine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Compounds having at least two amino groups may also be made from polyols such those described above. For example, hydroxyl groups of a polyol maybe reacted in an ester coupling reaction in the presence of a suitable coupling agent with a carboxyl group of a suitable amino acid compound in which the amino group of the amino acid is protected with a suitable protective group (e.g., a tert-butyloxycarbonyl (tBoc) protective group). Examples of amino acids may be selected, for example, from beta amino acids such as 3-aminopropanoic acid (also known as beta-alanine), gamma amino acids such as 4-aminobutanoic acid (also known as gamma-aminobutyric acid, or GABA), delta amino acids such as 5-aminopentanoic acid, epsilon amino acids such as 6-aminohexanoic acid, etc. Deprotection of the protective groups results in a compound in which a primary amine group is linked to a residue of the polyol through a hydrolysable ester group at the site of each of the hydroxyl groups of the polyol.

In some embodiments, the multifunctional compound is a compound having at two or more electrophilic groups, for example, having 2, 3, 4, 5, 6, 7, 8, 9, 10, 11, 12, 13, 14, 15, 16, 17, 18, 19, 20 or more electrophilic groups. Electrophilic groups may be selected, for example, from cyclic imide ester groups, such as succinimide ester groups, maleimide ester groups, glutarimide ester groups, diglycolimide ester groups, phthalimide ester groups, and bicyclo[2.2.1]hept-5-ene-2,3-dicarboxylic acid imide ester groups, imidazole ester groups, imidazole carboxylate groups and benzotriazole ester groups, among other possibilities.

Commercially available compounds that contain at least two cyclic imide ester groups include disuccinimidyl glutarate (containing 2 cyclic imide esters), 1,5-Bis(2,5-dioxo-1-pyrrolidinyl) 2-oxopentanedioate (containing 2 cyclic imide esters), 1,5-Bis(2,5-dioxo-1-pyrrolidinyl) 3-hydroxypentanedioate (containing 2 cyclic imide esters), 1,2,3-Tris(2,5-dioxo-1-pyrrolidinyl) 2-hydroxy-1,2,3-propanetricarboxylate (containing 3 cyclic imide esters).

Compounds that contain at least two cyclic imide ester groups can also be formed from compounds having at least two carboxylic acid groups along the lines described in conjunction with FIG. 3A by reaction with an N-hydroxy cyclic imide compound, such as one of those described above, in the presence of a coupling agent. Compounds having at least two carboxylic acid groups also include various known polycarboxylic acid compounds such as glutaric acid, succinic acid, malonic acid, adipic acid, diglycolic acid, citric acid, isocitric acid, trimellitic acid, trimesic acid, aconitic acid, propane-1,2,3-tricarboxylic acid (tricarballylic acid), ethanetricarboxylic acid, and butane-1,2,3,4-tetracarboxylic acid, and ethane-1,1,2,2-tetracarboxylic acid, among others. In a specific example, polycarboxylic acid compound is reacted with N-hydroxysuccinimide to form succinimidyl ester groups in the positions previously occupied by the carboxylic acid groups of the polycarboxylic acid compound.

Compounds having at least two carboxylic acid groups may also be made from polyols such those described above. For example, a polyol maybe reacted with a cyclic anhydride compound described above in a ring opening reaction to form a polycarboxylic acid compound in which each of the carboxylic acid groups are linked to a residue of the polyol through a hydrolysable ester group. Then, the polycarboxylic acid compound may be reacted with an N-hydroxy cyclic imide compound such as one of those described above in the presence of a coupling agent to form a compound in which cyclic imide ester groups are linked to a polyol residue through an ester linkage.

In some embodiments, the multifunctional compound is a compound having at least two strained alkyne groups, for example, having 2, 3, 4, 5, 6, 7, 8, 9, 10, 11, 12, 13, 14, 15, 16, 17, 18, 19, 20 or more strained alkyne groups. Strained alkyne groups may be selected, for example, from (1R,8S,9s)-bicyclo[6.1.0]non-4-yn-9-yl groups and dibenzocyclooctyne groups, among other possibilities.

Commercially available compounds that contain at least two strained alkyne groups include 5,8-Dioxa-2,11-diazadodecanedioic acid, 1,12-bis[(1R,8S)-bicyclo[6.1.0]non-4-yn-9-ylmethyl]ester, Poly(oxy-1,2-ethanediyl) (CAS 1476737-97-9, α-[2-[[[(1α,8α,9β)-bicyclo[6.1.0]non-4-yn-9-ylmethoxy]carbonyl]aminolethyl]-ω-[2-[[(1α,8α,9β)-bicyclo[6.1.0]non-4-yn-9-ylmethoxy]carbonyl]amino]ethoxy] (CAS 2152700-22-4), 2-(9-bicyclo[6.1.0]non-4-ynylmethoxy)-˜{N}-[5-[[2-(9-bicyclo[6.1.0]non-4-ynylmethoxy)acetyl]amino]pentyl]acetamide (CAS 2743204 May 7), 2-(9-bicyclo[6.1.0]non-4-ynylmethoxy)-˜{N}-[2-[2-[[2-(9-bicyclo[6.1.0]non-4-ynylmethoxy)acetyl]amino]ethoxy]ethyl]acetamide (CAS 2752158-76-0).

Compounds that contain at least two strained alkyne groups may also be formed from polycarboxylic acid compounds that contain at least two carboxylic acid groups.

Polycarboxylic acid compounds that contain at least two carboxylic acid groups include various known polycarboxylic acid compounds such as glutaric acid, succinic acid, malonic acid, adipic acid, diglycolic acid, citric acid, isocitric acid, trimellitic acid, trimesic acid, aconitic acid, propane-1,2,3-tricarboxylic acid (tricarballylic acid), ethanetricarboxylic acid, and butane-1,2,3,4-tetracarboxylic acid, and ethane-1,1,2,2-tetracarboxylic acid among others. Polycarboxylic acid compounds that contain at least two carboxylic acid groups may also be made from polyols as described above.

For example, carboxylic acid groups of a polycarboxylic acid compound may be reacted with a hydroxyl-substituted strained alkyne such as (1R,8S,9s)-bicyclo[6.1.0]non-4-yn-9-ylmethanol in an ester coupling reaction in the presence of a suitable coupling agent to produce (1R,8S,9s)-bicyclo[6.1.0]non-4-yn-9-yl groups that are coupled to a residue of the polycarboxylic acid compound through two hydrolysable ester groups. In an alternative embodiment, carboxyl groups of a polycarboxylic acid compound may be reacted with an amine-substituted strained alkyne such as (1R,8S,9s)-bicyclo[6.1.0]non-4-yn-9-ylmethamine in an amide coupling reaction in the presence of a suitable coupling agent to produce a compound in which (1R,8S,9s)-bicyclo[6.1.0]non-4-yn-9-yl groups are linked to a residue of the polycarboxylic acid compound through an amide group and a hydrolysable ester group.

In some embodiments, the multifunctional compound is a compound having at least two strained alkene groups, for example, having 2, 3, 4, 5, 6, 7, 8, 9, 10, 11, 12, 13, 14, 15, 16, 17, 18, 19, 20 or more strained alkene groups. Strained alkene groups may be selected, for example, from cyclooct-4-en-1-yl groups, cyclooct-4-enyl 2,5-dioxopyrrolidin-1-yl carbonate groups, carbamic acid, N-(3-aminopropyl)-, 4-cycloocten-1-yl ester groups, and carbamic acid, N-[2-[2-(2-aminoethoxy) ethoxy]ethyl]-, 4-cycloocten-1-yl ester groups, among other possibilities.

Commercially available compounds that contain at least two strained alkene groups include 5,8,11,14,17,20,23,26-octaoxa-2,29-diazatriacontanedioic acid, 1,30-di-4-cycloocten-1-yl ester, 5,8,11,14-tetraoxa-2,17-diazaoctadecanedioic acid, 1,18-di-4-cycloocten-1-yl ester, cyclooctene, 5,5′-[1,2-ethanediylbis(oxy-2,1-ethanediyloxy)]bis-, and cyclooctene, 5,5′,5″-[(methylsilylidyne)tris(oxy)]tris-

Compounds that contain at least two strained alkene groups may also be formed from polycarboxylic acid compounds that contain at least two carboxylic acid groups. For example, carboxylic acid groups of a polycarboxylic acid compound may be reacted with a hydroxyl-substituted strained alkene such as cyclooct-4-en-1-ol in an ester coupling reaction in the presence of a suitable coupling agent to produce a compound in which cyclooct-4-en-1-yl groups are linked to a residue of the polycarboxylic acid compound through two hydrolysable ester groups. In an alternative embodiment, carboxyl groups of a polycarboxylic acid compound may be reacted with an amine-substituted strained alkene such as cyclooct-4-en-1-amine in an amide coupling reaction in the presence of a suitable coupling agent to produce a compound in which cyclooct-4-en-1-yl groups are linked to a residue of the polycarboxylic acid compound through an amide group and a hydrolysable ester group.

Polycarboxylic acid compounds that contain at least two carboxylic acid groups include various known polycarboxylic acid compounds such as those described above. Polycarboxylic acid compounds that contain at least two carboxylic acid groups may also be made from polyols as described above.

In some embodiments, the multifunctional compound is a compound having at least two tetrazine groups, for example, having 2, 3, 4, 5, 6, 7, 8, 9, 10, 11, 12, 13, 14, 15, 16, 17, 18, 19, 20 or more tetrazine groups.

Commercially available compounds that contain at least two tetrazine groups include 5,5′-Bi-1,2,3,4-tetrazine,

Compounds that contain at least two tetrazine groups may also be formed from polyols that that contain at least two hydroxyl groups. Exemplary polyols include those described above for use in forming multi-arm polymers.

In some embodiments, a tetrazine based acid may be coupled to hydroxyl groups of a polyol that that contains at least two hydroxyl groups in an ester coupling reaction in the presence of a suitable coupling agent. In a particular example, multifunctional compound containing tetrazine groups may be prepared by coupling hydroxyl groups of a polyol with 5-[4-(1,2,4,trazin-3-yl)benzylamino]-5-oxopentanoic acid in the presence of a carbodiimide coupling agent to produce a compound in which 5-[4-(1,2,4,5-tetrazin-3-yl)benzylamino]-5-oxopentanoate groups are linked to a polyol residue at a site of each of the hydroxyl groups of the precursor polyol. It is noted that each 5-[4-(1,2,4,5-tetrazin-3-yl)benzylamino]-5-oxopentanoate group contains a hydrolysable ester group.

In some embodiments, the multifunctional compound is a compound having at least two azide groups, for example, having 2, 3, 4, 5, 6, 7, 8, 9, 10, 11, 12, 13, 14, 15, 16, 17, 18, 19, 20 or more azide groups.

Commercially available compounds that contain at least two azide groups include 1,1′-oxybis[2-(2-azidoethoxy) ethane], 1,17-diazido-3,6,9,12,15-pentaoxaheptadecane, N,N′-(dithiodi-2,1-ethanediyl)bis[4-azido-2-hydroxybenzamide], propane, 1-azido-3-[(2-azidoethoxy) methoxy]-, and 1,3,5-tris(azidomethyl)-2,4,6-triethylbenzene.

Multifunctional compounds having at least two azide groups may be formed from polyols having at least two hydroxyl groups. Exemplary polyols include those described above for use in forming multi-arm polymers.

For example, a multifunctional compound having at least two azide groups may be synthesized by reacting hydroxyl groups of a polyol with methanesulfonyl chloride to obtain multifunctional compound having at least two methanesulfonyl groups. Then, sodium azide (NaN₃) is reacted with the multifunctional compound having at least two methanesulfonyl groups to replace the methanesulfonate groups with azide groups, thereby forming a multifunctional compound having at least two azide groups.

Polyols having at least two hydroxyl groups and a biodegradable ester group may also be made from polyols such those described above for use in forming multi-arm polymers. For example, a precursor polyol may be reacted in a ring-opening reaction with a lactone (e.g., α-acetolactone, β-propiolactone, γ-butyrolactone, δ-valerolactone, ε-caprolactone, etc.) to form a further polyol that comprises a hydroxyl end group that is linked to a residue of precursor polyol through an alkyl group (e.g., a C₁-C₁₀ alkyl group) and a hydrolysable ester group at the site of each of the hydroxyl groups of polyol. This polyol may be used to form a multifunctional compound having at least two azide groups, as described in the prior paragraph. The at least two azide groups will be linked to a remainder of the multifunctional

As previously indicated, in addition to a reactive polymer comprising a plurality of first hydrophilic polymer segments and a plurality of first reactive moieties and a reactive multifunctional compound comprising a plurality of second reactive moieties, the systems described herein further comprise gold nanoparticles that are functionalized with a plurality of second hydrophilic polymer segments. The first and second hydrophilic polymer segments may be formed from the same monomers or different monomers.

The gold nanoparticles may be solid gold particles, or the gold nanoparticles may comprise a gold shell surrounding a core particle that is formed from a metal other than gold, for example, tantalum or tungsten, among others. The gold in the nanoparticles may be substantially pure gold or may by a gold alloy (e.g., gold alloyed with one or more metals selected from silver, platinum, copper, titanium, rhodium, palladium, zinc, nickel, iron, and aluminum).

The gold nanoparticles may be provided in a variety of shapes, including spherical, rod-shaped, plate-shaped and irregularly shaped. The size of the gold nanoparticle may be chosen for the specific imaging application and the desired radiopacity. See Xi, D. et al., Gold nanoparticles as computerized tomography (CT) contrast agents. RSC Adv. 2012, 2 (33), 12515-12524. For example, where the imaging is X-ray based, the particles may range from 1 nm to 2000 nm, for example, ranging anywhere from 1 nm to 2 nm to 5 nm to 10 nm to 20 nm to 50 nm to 100 nm to 200 nm to 500 nm to 1000 nm to 2000 nm, in longest dimension (e.g., diameter for a sphere, length for a rod, greatest width for a platelet, etc.). As another example, where the imaging is near-IR fluorescence spectrometry-based, the particles may range from 20 nm to 500 nm, for example, ranging anywhere from 20 nm to 50 nm to 100 nm to 200 nm to 500 nm in longest dimension.

Referring now to FIG. 2, a functionalized gold nanoparticle 216 in accordance with the present disclosure is shown that comprises a plurality of polymer chains linked to a gold nanoparticle 212, the polymer chains comprising a hydrophilic polymer segment 214. One end of the hydrophilic polymer segment 214 is covalently linked to the gold nanoparticle 212.

Gold nanoparticles may be surface functionalized with a hydrophilic polymer that comprises a hydrophilic polymer segment 214 comprising a thiol group (—SH group) at one end. Surface functionalization of the gold nanoparticles occurs readily when a thiol terminated polymer is added to a solution of gold nanoparticles. See, e.g., Gao, J. et al., Colloidal Stability of Gold Nanoparticles Modified with Thiol Compounds: Bioconjugation and Application in Cancer Cell Imaging. Langmuir 2012, 28 (9), 4464-4471; Zopes, D. et al., Improved Stability of “Naked” Gold Nanoparticles Enabled by in Situ Coating with Mono and Multivalent Thiol PEG Ligands. Langmuir 2013, 29 (36), 11217-11226; Hinterwirth, H. et al., Quantifying Thiol Ligand Density of Self-Assembled Monolayers on Gold Nanoparticles by Inductively Coupled Plasma-Mass Spectrometry. ACS Nano 2013, 7 (2), 1129-1136.

In some embodiments, after surface functionalization by reaction with a thiol-terminated hydrophilic polymer that comprises a hydrophilic polymer segment and a thiol group, the functionalized gold nanoparticles may be surface-passivated to prevent further reaction of the gold surface, for example, with the reactive multifunctional compounds described herein. For example, the gold surface may be passivated by citrate ligands. The citrate ligands will be displaced by the thiol-terminated hydrophilic polymers; however, some citrate ligands may remain. The ratio of thiol-terminated hydrophilic polymers to citrate ligands (or other ligands) may be varied to optimize the process.

Hydrophilic polymer segments for use in forming functionalized gold nanoparticles can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, 8-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, among others, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, or polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the functionalized gold nanoparticles of the present disclosure typically contain between 5 or less and 100 or more monomer units or more, for example, ranging from 2 to 5 to 10 to 20 to 50 to 100 to 200 monomer units in length.

In various embodiments, the polymer segments of the functionalized gold nanoparticles of the present disclosure are formed from the same monomers as the polymer segments of the reactive polymer.

As previously indicated, in some aspects, the present disclosure provides a radiopaque hydrogel that comprises a crosslinked reaction product of (a) a reactive polymer as described herein and (b) a reactive multifunctional compound as described herein. In various embodiments, the reactive polymer will have three or more first reactive end groups (e.g., three or more polymer arms terminated with a first reactive moiety R₁) and the multifunctional compound will have two or more second reactive end groups.

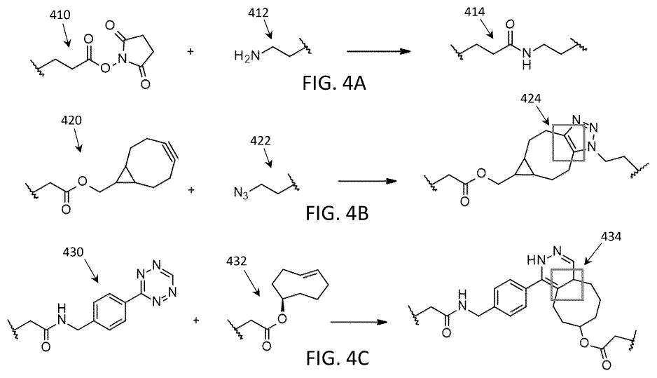

Three specific examples of covalent crosslinking reactions between first and second reactive moieties are shown in FIGS. 4A-4C.

FIG. 4A shows a covalent crosslinking reaction between a cyclic amide ester group, specifically, a succinimide ester group 410 and a primary amine group 412, whereby an amide linking group 414 is formed. In this scheme, the first reactive moiety may comprise the cyclic amide ester group and the second reactive moiety may comprise the primary amine group, or the first reactive moiety may comprise the primary amine group and the second reactive moiety may comprise the cyclic amide ester group.

FIG. 4B shows the formation of a cyclooctatriazole covalent linkage 424 through a strain-promoted azide-alkyne cycloaddition click chemistry (SPACC) reaction between a strained alkyne group, specifically, a (1R,8S,9s)-bicyclo[6.1.0]non-4-yn-9-yl-ester group 420 with an azide group 422. The box in FIG. 4B denotes where the new covalent bonding is formed. In this scheme, the first reactive moiety may comprise the strained alkyne group and the second reactive moiety may comprise the azide group, or the first reactive moiety may comprise the azide group and the second reactive moiety may comprise the strained alkyne group.

FIG. 4C shows the formation of a cyclooctapyridazine covalent linkage 434 through a strain-promoted tetrazine ligation coupling reaction between a tetrazine group, specifically, a 1,2,4,5-tetrazin-3-yl group 430 and a strained alkene group, specifically, a cyclooct-4-en-1-yl-ester group 432. The box in FIG. 4C denotes where the new covalent bonding is formed. In this scheme, the first reactive moiety may comprise the strained alkene group and the second reactive moiety may comprise the tetrazine group, or the first reactive moiety may comprise the tetrazine group and the second reactive moiety may comprise the strained alkene group.

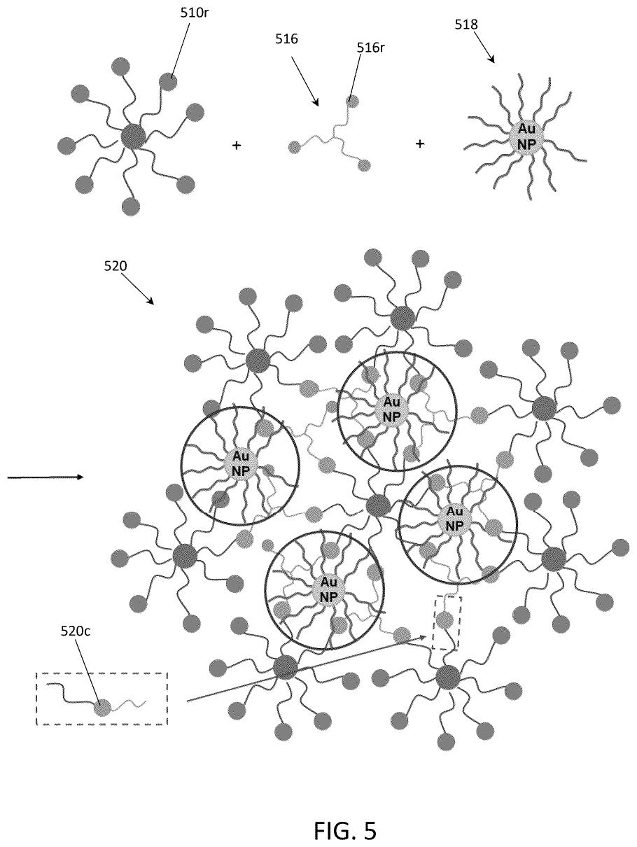

An overall crosslinking reaction is illustrated schematically in FIG. 5, which shows a reactive multi-arm polymer 510 having a plurality of first reactive moieties (designated by circles 510r) being covalently crosslinked with reactive multifunctional compound 516 having a plurality of reactive moieties (designated by circles 515r) in the presence of functionalized gold nanoparticles 518 to form a crosslinked reaction product, specifically, a crosslinked radiopaque hydrogel 520, in which covalent bonds (designated by circles 520c) are formed between the reactive multi-arm polymer 510 and the reactive multifunctional compound 516.

In various embodiments, the functionalized gold nanoparticles are homogeneously mixed with the reactive multi-arm polymer and/or the reactive multifunctional compound prior to hydrogel formation. Once the hydrogel is formed, the functionalized gold nanoparticles are locked into the hydrogel via non-covalent interactions (such as chain entanglement and/or hydrophilic interactions such as hydrogen bonding) between hydrophilic polymer segments in the hydrophilic polymer chains that are attached to the surfaces of the functionalized gold nanoparticles and hydrophilic polymer segments in the polymer arms of the multi-arm polymers that are covalently incorporated into the hydrogel.

The radiopacity of the hydrogel may be tuned, for example, by modifying the concentration of the functionalized gold nanoparticles within the hydrogel. In some embodiments, the concentration of the functionalized gold nanoparticles within the hydrogel ranges from 0.01 mg/mL or less to 100 mg/mL or more, for example, ranging anywhere from 0.01 mg/mL to 0.02 mg/mL to 0.05 mg/mL to 0.1 mg/mL to 0.2 mg/mL to 0.5 mg/mL to 1 mg/mL to 2 mg/mL to 5 mg/mL to 10 mg/mL to 20 mg/mL to 50 mg/mL to 100 mg/mL.

In some embodiments, the present disclosure provides hydrogels that comprise functionalized gold nanoparticles and a crosslinked reaction product of a reactive polymer comprising first reactive moieties that comprise electrophilic groups and a multifunctional compound comprising second reactive moieties that comprise nucleophilic groups. In some embodiments, the present disclosure provides hydrogels that comprise functionalized gold nanoparticles and a crosslinked reaction product of a reactive polymer comprising first reactive moieties that comprise nucleophilic groups and a multifunctional compound comprising second reactive moieties that comprise electrophilic groups.

In some embodiments, the present disclosure provides a system for forming hydrogels by combining the following in a reaction mixture: functionalized gold nanoparticles, a reactive polymer comprising first reactive moieties that comprise electrophilic groups, and a multifunctional compound comprising second reactive moieties that comprise nucleophilic groups. In some embodiments, the present disclosure provides a system for forming hydrogels by combining the following in a reaction mixture: functionalized gold nanoparticles, a reactive polymer comprising first reactive moieties that comprise nucleophilic groups, and a multifunctional compound comprising second reactive moieties that comprise electrophilic groups. The functionalized gold nanoparticles, reactive polymer and the multifunctional compound are combined under conditions such that the electrophilic and nucleophilic groups crosslink with one another, forming the hydrogel and trapping the functionalized gold nanoparticles in the hydrogel. In certain embodiments, those conditions comprise an environment having a basic pH, for example, a pH ranging from about 9 to about 11. Such hydrogels can be formed in vivo or ex vivo.

In some embodiments, the present disclosure provides hydrogels that comprise functionalized gold nanoparticles and a crosslinked reaction product of a reactive polymer comprising first reactive moieties that comprise strained alkyne groups and a multifunctional compound comprising second reactive moieties that comprise azide groups. In some embodiments, the present disclosure provides hydrogels that comprise functionalized gold nanoparticles and a crosslinked reaction product of a reactive polymer comprising first reactive moieties that comprise azide groups and a multifunctional compound comprising second reactive moieties that comprise strained alkyne groups.

In some embodiments, the present disclosure provides a system for forming hydrogels by combining the following in a reaction mixture: functionalized gold nanoparticles, a reactive polymer comprising first reactive moieties that comprise strained alkyne groups, and a multifunctional compound comprising second reactive moieties that comprise azide groups. In some embodiments, the present disclosure provides a system for forming hydrogels by combining the following in a reaction mixture: functionalized gold nanoparticles, a reactive polymer comprising first reactive moieties that comprise azide groups, and a multifunctional compound comprising second reactive moieties that comprise strained alkyne groups. The reactive polymer and the multifunctional compound are combined under conditions such that the strained alkyne and azide groups crosslink with one another, forming the hydrogel and trapping the functionalized gold nanoparticles in the hydrogel. Such hydrogels can be formed in vivo or ex vivo.

In some embodiments, the present disclosure provides hydrogels that comprise functionalized gold nanoparticles and a crosslinked reaction product of a reactive polymer comprising first reactive moieties that comprise strained alkyne groups and a multifunctional compound comprising second reactive moieties that comprise tetrazine groups. In some embodiments, the present disclosure provides hydrogels that comprise functionalized gold nanoparticles and a crosslinked reaction product of a reactive polymer comprising first reactive moieties that comprise tetrazine groups and a multifunctional compound comprising second reactive moieties that comprise strained alkyne groups.

In some embodiments, the present disclosure provides a system for forming hydrogels that comprise a crosslinked reaction product by combining the following in a reaction mixture: functionalized gold nanoparticles, a reactive polymer comprising first reactive moieties that comprise strained alkyne groups, and a multifunctional compound comprising second reactive moieties that comprise tetrazine groups. In some embodiments, the present disclosure provides a system for forming hydrogels that comprise a crosslinked reaction product by combining the following in a reaction mixture: functionalized gold nanoparticles, a reactive polymer comprising first reactive moieties that comprise tetrazine groups, and a multifunctional compound comprising second reactive moieties that comprise strained alkyne groups. The reactive polymer and the multifunctional compound are combined under conditions such that the strained alkyne and tetrazine groups crosslink with one another, forming the hydrogel and trapping the functionalized gold nanoparticles in the hydrogel. Such hydrogels can be formed in vivo or ex vivo.

In various embodiments, the hydrogels of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values) for example, when measured on a bench-top micro-CT system such as XtremeCT from Scanco Medical (Wangen-Brüttisellen, Switzerland) or similar.

The hydrogels of the present disclosure can be used in a wide variety of biomedical applications, including implants, medical devices, and pharmaceutical compositions.

In some aspects of the present disclosure, a system is provided that comprises (a) a first composition that comprises a reactive polymer as described herein and (b) a second composition that comprises multifunctional compound as described herein, wherein the first composition, the second composition, or both, comprises functionalized gold nanoparticles as described herein, and wherein when the first and second compositions are combined, covalent crosslinks form between the reactive polymer and the multifunctional compound, creating a hydrogel and trapping the functionalized gold nanoparticles in the hydrogel.

The first composition may be a first fluid composition comprising the multifunctional compound or a first dry composition that comprises the multifunctional compound, to which a suitable fluid such as water for injection, saline, etc. can be added to form a first fluid composition. In addition to the multifunctional compound, the first composition may further comprise functionalized gold nanoparticles and/or additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise functionalized gold nanoparticles and/or additional agents, including therapeutic agents, imaging agents, colorants, tonicity adjusting agents, suspension agents, wetting agents, and pH adjusting agents as described below.

In some embodiments, the system is configured to combine a first fluid composition comprising the multifunctional compound with a second fluid comprising the reactive polymer, wherein the first composition, the second composition, or both, comprises functionalized gold nanoparticles as described herein, and wherein the first composition, the second composition, or both, may optionally comprise additional agents as described herein. Upon mixing the first and second fluid compositions, the multifunctional compound crosslink with the reactive polymer, forming a hydrogel and trapping the functionalized gold nanoparticles in the hydrogel. The first and second fluid compositions may be combined to form radiopaque crosslinked hydrogels, either in vivo or ex vivo.

In some embodiments, the multifunctional compound is initially combined with the reactive polymer under conditions where crosslinking between the reactive polymer and the multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the multifunctional compound and the reactive polymer, thereby forming a crosslinked product.

In some embodiments, the system comprises (a) a first composition that comprises a multifunctional compound as described herein and a reactive polymer as described herein and (b) a second composition, specifically, an accelerant composition, that contains an accelerant that is configured to accelerate a crosslinking reaction between the multifunctional compound and the reactive polymer. In some embodiments, the first composition may further comprise functionalized gold nanoparticles and/or additional agents as described herein. In some embodiments, the second composition may further comprise functionalized gold nanoparticles and/or additional agents as described herein. In some embodiments, the system may comprise a third composition that comprises functionalized gold nanoparticles as described herein.

In some embodiments, the system comprises (a) a first composition that comprises multifunctional compound as described herein, (b) a second composition that comprises a reactive polymer as described herein, and (c) a third composition, specifically, an accelerant composition, that contains an accelerant that is configured to accelerate a crosslinking reaction between the multifunctional compound and the reactive polymer. In some embodiments, the first composition may further comprise functionalized gold nanoparticles and/or additional agents as described herein. In some embodiments, the second composition may further comprise functionalized gold nanoparticles and/or additional agents as described herein. In some embodiments, the third composition may further comprise functionalized gold nanoparticles and/or additional agents as described herein. In some embodiments, the system may comprise a fourth composition that comprises functionalized gold nanoparticles as described herein.

The first composition may be a first fluid composition comprising the multifunctional compound that is buffered to an acidic pH or a first dry composition that comprises the multifunctional compound, to which a suitable fluid such as water for injection, saline, an acidic buffer solution, etc. can be added to form a first fluid composition comprising the multifunctional compound that is buffered to an acidic pH. In some embodiments, for example, the acidic buffering composition may comprise monobasic sodium phosphate, among other possibilities. The first fluid composition comprising the multifunctional compound may have a pH ranging, for example, from about 3 to about 5. In addition to the multifunctional compound, the first composition may further comprise functionalized gold nanoparticles and/or additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the multifunctional compound that is buffered to an acidic pH. In addition to the reactive polymer, the second composition may further comprise functionalized gold nanoparticles and/or additional agents, including therapeutic agents, imaging agents, colorants, tonicity adjusting agents, suspension agents, wetting agents, and pH adjusting agents as described below.

In a particular embodiment, the first composition is a first fluid composition comprising the multifunctional compound (as well as functionalized gold nanoparticles and/or additional agents in some cases) that is buffered to an acidic pH and the second composition is a dry composition that comprises the reactive polymer (as well as functionalized gold nanoparticles and/or additional agents in some cases). The first composition may then be mixed with the second composition to provide a prepared fluid composition that is buffered to an acidic pH and comprises the multifunctional compound and the reactive polymer. In a particular example, a syringe may be provided that contains the first fluid composition comprising the multifunctional compound that is buffered to an acidic pH, and a vial may be provided that comprises the second dry composition (e.g., a powder) that comprises the reactive polymer. The syringe may then be used to inject the first fluid composition into the vial containing the reactive polymer to form a prepared fluid composition that is buffered to an acidic pH and contains the multifunctional compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise functionalized gold nanoparticles and/or additional, including those described below.

A prepared fluid composition that is buffered to an acidic pH and comprises the multifunctional compound and the reactive polymer as described above (as well as functionalized gold nanoparticles and/or additional agents in some cases), and a fluid accelerant composition that is buffered to basic pH as described above (which may include functionalized gold nanoparticles and/or additional agents in some cases), may be combined form radiopaque crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd (III), Mn (II), Fc (III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles (beyond the functionalized gold particles that are present) such as carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fc, 42K, 82Rb, 24Na, 45Ti, 44Sc, 51Cr and 177Lu, among others, and (f) radiocontrast agents (beyond the functionalized gold particles that are present), for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises multifunctional compound as described above and a second reservoir that contains a second fluid composition that comprises a reactive polymer as described above, wherein the first and second fluid compositions form a crosslinked product upon mixing. As previously noted, the first fluid composition, the second fluid composition, or both, comprises functionalized gold nanoparticles as described herein. Moreover, the first fluid composition, the second fluid composition, or both, may optionally comprise additional agents as described herein.

In some embodiments, the system may include a delivery device that comprises a first reservoir that contains a first fluid composition that comprises the multifunctional compound and the reactive polymer and is buffered to an acidic pH, such as the prepared fluid composition previously described, and a second reservoir that contains second fluid composition, such as the fluid accelerant composition previously described. As previously noted, the first fluid composition, the second fluid composition, or both, comprises functionalized gold nanoparticles as described herein. Moreover, the first fluid composition, the second fluid composition, or both, may optionally comprise additional agents as described herein.

In either case, during operation, the first fluid composition and second fluid composition are dispensed from the first and second reservoirs and combined, whereupon the multifunctional compound and the reactive polymer and crosslink with one another to form a radiopaque crosslinked hydrogel.

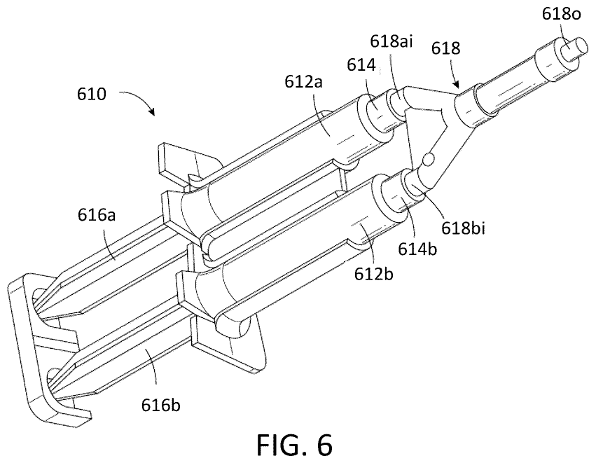

In particular embodiments, and with reference to FIG. 6, the system may include a delivery device 610 that comprises a double-barrel syringe, which includes first barrel 612a having a first barrel outlet 614a, which first barrel contains the first composition, a first plunger 616a that is movable in the first barrel 612a, a second barrel 612b having a second barrel outlet 614b, which second barrel 612b contains the second composition, and a second plunger 616b that is movable in the second barrel 612b. In some embodiments, the device 610 may further comprise a mixing section 618 having a first mixing section inlet 618ai in fluid communication with the first barrel outlet 614a, a second mixing section inlet 618bi in fluid communication with the second barrel outlet, and a mixing section outlet 618o.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a radiopaque crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Radiopaque crosslinked hydrogel particles formed using the above and other techniques may varying widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

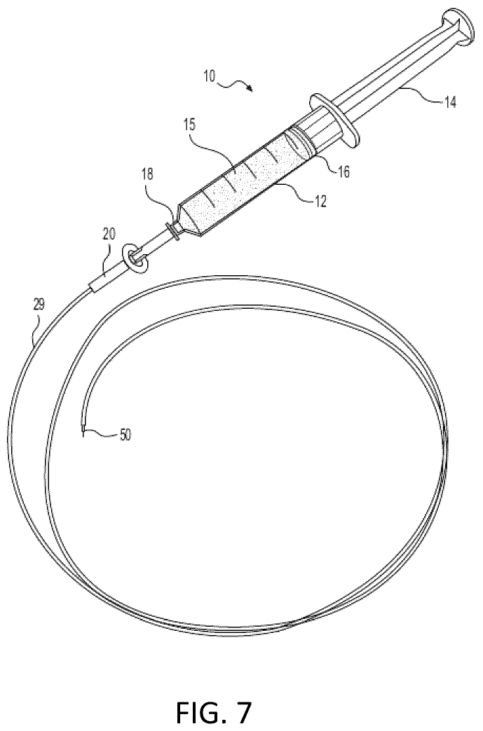

FIG. 7 illustrates a syringe 10 providing a reservoir for a radiopaque crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a radiopaque crosslinked hydrogel composition 15 for injection through the needle 50.

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

During and/or after administration, the radiopaque crosslinked hydrogel compositions of the present disclosure can be imaged using a suitable imaging technique.

As seen from the above, the radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A system comprising (a) a reactive polymer comprising a plurality of first hydrophilic polymer segments and a plurality of first reactive moieties, (b) a reactive multifunctional compound comprising a plurality of second reactive moieties, and (c) gold nanoparticles that are functionalized with a plurality of second hydrophilic polymer segments.

2. The system of claim 1, wherein the reactive polymer is a multi-arm polymer that comprises three or more polymer arms linked to a core region, each arm comprising one of the first hydrophilic polymer segments and one of the first reactive moieties.

3. The system of claim 1, wherein the reactive polymer is a multi-arm polymer that comprises three or more polymer arms linked to a core region, each arm comprising a cyclic anhydride residue disposed between a first hydrophilic polymer segment and a first reactive moiety.

4. The system of claim 1, wherein the first hydrophilic polymer segments are selected from poly(alkylene oxide) segments, polysaccharide segments, polyoxazoline segments, polydioxanone segments, polypeptide segments, and polyvinyl alcohol segments.

5. The system of claim 1, wherein each of the first hydrophilic polymer segments contains between 10 and 1000 monomer residues.

6. The system of claim 1, wherein the gold nanoparticles range from 1 nm to 2 micrometers in longest dimension.

7. The system of claim 1, wherein the second hydrophilic polymer segments is selected from poly(alkylene oxide) segments, polysaccharide segments, polyoxazoline segments, polydioxanone segments, polypeptide segments, and polyvinyl alcohol segments.

8. The system of claim 1, wherein the second reactive moieties is attached to a polyol residue or a polycarboxylic acid residue.

9. The system of claim 1, wherein the second reactive moieties is attached to the multifunctional compound through a hydrolysable ester group.

10. The system of claim 1, wherein the first reactive moieties comprises a cyclic imide ester group and the second reactive moieties comprises a primary amine, thiol or hydroxyl group, or wherein the first reactive moieties comprises a primary amine, thiol or hydroxyl group and the second reactive moieties comprises a cyclic imide ester group.

11. The system of claim 1, wherein the first reactive moieties comprises a strained alkyne group and the second reactive moieties comprises an azide group, or wherein the first reactive moieties comprises an azide group and the second reactive moieties comprises a strained alkyne group.

12. The system of claim 1, wherein the first reactive moieties comprises a strained alkene group and the second reactive moieties comprises tetrazine a group, or wherein the first reactive moieties comprises a tetrazine group and the second reactive moieties comprises a strained alkene group.

13. The system of claim 1, further comprising a delivery device.

14. A method of treatment comprising administering to a subject a mixture that comprises a reactive polymer comprising a plurality of first hydrophilic polymer segments and a plurality of first reactive moieties, a reactive multifunctional compound comprising a plurality of second reactive moieties, and gold nanoparticles that are functionalized with a plurality of second hydrophilic polymer segments, under conditions such that the reactive polymer and the multifunctional compound crosslink after administration to form a crosslinked hydrogel in which the gold nanoparticles are retained in the crosslinked hydrogel via non-covalent interactions.

15. The method of claim 14, wherein the method comprises administering to the subject a first fluid composition that comprises the reactive polymer and a second fluid composition that comprises the multifunctional compound, wherein the gold nanoparticles are provided in the first fluid composition, the second composition, or both.

16. The method of claim 14, wherein the method comprises administering to the subject a first fluid composition that comprises the reactive polymer and the multifunctional compound and a second fluid composition that comprises an accelerant that accelerates formation of the covalent crosslinks, and wherein the gold nanoparticles are provided in the first fluid composition, the second composition, or both.

17. The method of claim 14, wherein the first and second hydrophilic polymer segments are selected from poly(alkylene oxide) segments, polysaccharide segments, polyoxazoline segments, polydioxanone segments, polypeptide segments, and polyvinyl alcohol segments.

18. A crosslinked hydrogel comprising gold nanoparticles retained via non-covalent interactions in a crosslinked reaction product of (a) a reactive polymer comprising a plurality of first hydrophilic polymer segments and a plurality of first reactive moieties and (b) a reactive multifunctional compound comprising a plurality of second reactive moieties, wherein the gold nanoparticles are functionalized with a plurality of second hydrophilic polymer segments.

19. The crosslinked hydrogel of claim 18, wherein the crosslinked hydrogel is in the form of injectable particles.

20. The crosslinked hydrogel of claim 18, wherein the first and second hydrophilic polymer segments are selected from poly(alkylene oxide) segments, polysaccharide segments, polyoxazoline segments, polydioxanone segments, polypeptide segments, and polyvinyl alcohol segments.
